(19) United States
(12) Patent Application Publication
(10) Pub. No.: US 2014/0202485 A1
(43) Pub. Date: Jul. 24, 2014

(54) PERSONAL CARE COMPOSITION AND METHODS INCORPORATING LOW GELATION TEMPERATURE METHYLCELLULOSE

(71) Applicants: Miao Wang, Schwenksville, PA (US); Susan L. Jordan, Doylestown, PA (US)

(72) Inventors: Miao Wang, Schwenksville, PA (US); Susan L. Jordan, Doylestown, PA (US)

(73) Assignees: UNION CARBIDE CHEMICALS & PLASTICS TECHNOLOGY LLC, Midland, MI (US); ROHM AND HAAS COMPANY, Philadelphia, PA (US)

(21) Appl. No.: 14/235,577

(22) PCT Filed: Sep. 14, 2012
(86) PCT No.: PCT/US2012/055383

§ 371 Date: Jan. 28, 2014

Related U.S. Application Data
(60) Provisional application No. 61/540,843, filed on Sep. 29, 2011.

(51) Int. Cl. A61K 8/73; A61K 8/81; A61Q 5/06; A61Q 19/08; A61Q 17/04; A61Q 5/12; A61Q 5/00; A61Q 19/00

(52) U.S. Cl. CPC A61K 8/731; A61Q 5/00; A61K 8/8152; A61Q 19/00; A61Q 19/08; A61Q 17/04; A61Q 5/12; A61Q 5/06
USPC 132/206; 424/70.13; 424/47; 424/59; 424/78.03

(57) ABSTRACT

Described are personal care compositions, comprising water, a methylcellulose that gels at 45C or less, and at least one one hair fixative polymer, moisturizer, conditioner, humectant, cationic conditioning polymer, anti-aging active, or sun care active.

### FIELD

The present invention relates to personal care compositions.

### BACKGROUND

Personal care compositions must be effective at delivering active ingredients (such as sunscreens, moisturizers, styling polymers, conditioning polymers, and others), while still having good aesthetic properties (tactile, visual, and the like). In fact, aesthetic properties are of paramount importance in personal care, because a consumer associates these properties with performance and value.

When it comes to lotions and creams, a light, fluffy, airy texture is highly valued by consumers. As can be appreciated, it is difficult to incorporate the necessary active ingredients and create such a texture, it is more difficult still to maintain such a texture through the variable and sometimes extreme conditions faced by the product during packaging, transporting, and vending before it arrives in the hands of the consumer. Even were a desirable texture obtainable, traditional rheology modifiers, such as ASE (alkali-soluble emulsion), HASE (Hydrophobically modified alkali-soluble emulsion), HEC (Hydroxyethyl Cellulose), starch, clay, or other natural polymers, do not preserve desirable texture upon exposure to high temperature (also known as heat-aging).

Accordingly, it would be desirable to find new materials to impart improved aesthetics to personal care compositions that also provide stability over time and exhibit a reduced degree of thinning.

### DETAILED DESCRIPTION

In one embodiment, the present invention provides personal care compositions, comprising water, a methylcellulose that gels at 45° C. or less, and at least one one hair fixative polymer, moisturizer, conditioner, humectant, cationic conditioning polymer, anti-aging active, or sun care active.

Cellulose has a polymeric backbone repeating structure of anhydroglucose units joined by 1-4 linkages. Each anhydroglucose unit contains hydroxyl groups at the 2, 3, and 6 positions. Substitution of these hydroxyls creates cellulose derivatives. For example, treatment of cellulosic fibers with caustic solution, followed by a methylating agent, yields cellulose ethers substituted with one or more methoxy groups. If not further substituted with other alkyls, this cellulose derivative is known as methylcellulose.

Methylcellulose typically has a weight average molecular weight of at least 12 kDa, preferably at least 15 kDa. A rough description of a particular methylcellulose can be given by its “DS,” a term that refers to the average degree of methoxyl substitution per anhydroglucose unit of the cellulose derivative. Theoretically, methylcellulose can have a DS of 1 to 3, but in practice, methylcellulose typically has a DS of 1.47 to 2.2.

Conventionally, methylcellulose has been found to be very useful in a variety of applications, providing thickening, freeze/thaw stability, lubricity, moisture retention and release, film formation, texture, consistency, shape retention, emulsification, binding, gelation, and suspension properties.

One unusual property of methylcellulose is that it is known to exhibit reverse thermal gelation, in other words, methylcellulose gels at warmer temperatures and forms a liquid at cooler temperatures. Most grades of methylcellulose gel at around 50° C. to 60° C. A grade of methylcellulose that gels at a relatively low temperature, 38° C. to 44° C., is generally available under the tradename METHOCEL SG or SGA (The Dow Chemical Company). No grades of commercially available methylcellulose gel at temperatures as low as an individual's normal body temperature, however, U.S. Pat. No. 6,235,893, the entirety of which is incorporated by reference herein, teaches methylcelluloses that gel as low as 31° C.

In a preferred embodiment, the gelation is temperature-activated by a customer's body temperature, i.e., no crosslinker is required. In a preferred embodiment, the present methylcellulose is made according to the processes described in U.S. Pat. No. 6,235,893. U.S. Pat. No. 6,235,893 described lower gelation temperature as a desired and preferred but non-essential feature, (col. 3, lines 32-33). In contrast, however, as can be appreciated, a gelation temperature at or below the individual's body temperature is a critical feature for the present application in embodiments where the gelation is temperature-activated by the individual's body temperature.

In practice, the liquid should contain sufficient methylcellulose to induce the proper rate of gelation and strength of gel, as well as to achieve an initial viscosity (before imbibition) of at least 600 mPa·s, preferably at least 1000 mPa·s when measured at a shear rate of 10 sec-1. Accordingly, concentrations of methylcellulose in the liquid may understandably vary.
Methods of making methylcellulose are described in detail in U.S. Pat. No. 6,235,893. Generally, cellulose pulp is treated with a caustic, for example an alkali metal hydroxide. Preferably, 1 to 3.5 mol NaOH per mole of anhydroglucose units in the cellulose is used. Uniform swelling and alkali distribution in the pulp is optionally controlled by mixing and agitation. The rate of addition of aqueous alkaline hydroxide is governed by the ability to cool the reactor during the exothermic alkalization reaction. In one embodiment, an organic solvent such as dimethyl ether is added to the reactor as a diluent and a coolant. Likewise, the headspace of the reactor is optionally purged with an inert gas (such as nitrogen) to control oxygen-catalyzed depolymerization of the cellulose ether product. In one embodiment, the temperature is maintained at or below 45° C.

A methylating agent, such as methyl chloride or dimethyl sulfate, is also added by conventional means to the cellulose pulp, either before, after, or concurrent with the caustic, generally in an amount of 1.5 to 4 mol methylating agent per mole of anhydroglucose units in the cellulose. Preferably, the methylating agent is added after the caustic. Once the cellulose has been contacted with caustic and methylating agent, the reaction temperature is increased to 75° C. and reacted at this temperature for about half an hour.

In a preferred embodiment, a staged addition is used, i.e., a second amount of caustic is added to the mixture over at least 60 minutes, preferably at least 90 minutes, while maintaining the temperature at least 55° C., preferably a least 65° C., more preferably at least 80° C. Preferably, 2 to 4 mol caustic per mole of anhydroglucose units in the cellulose is used. A staged second amount of methylating agent is added to the mixture, either before, after, or concurrent with the caustic, generally in an amount of 2 to 4.5 mol methylating agent per mole of anhydroglucose units in the cellulose.

The cellulose ether is washed to remove salt and other reaction by-products. Any solvent in which salt is soluble may be employed, but water is preferred. The cellulose ether may be washed in the reactor, but is preferably washed in a separate washer located downstream of the reactor. Before or after washing, the cellulose ether may be stripped by exposure to steam to reduce residual organic content.

The cellulose ether is dried to a reduced moisture and volatile content of preferably 0.5 to 10.0 weight percent water and more preferably 0.8 to 5.0 weight percent water and volatiles based upon the weight of cellulose ether. The reduced moisture and volatiles content enables the cellulose ether to be milled into particulate form. The cellulose ether is milled to particulates of desired size. If desired, drying and milling may be carried out simultaneously.

In one embodiment, the personal care composition further comprises one or more rheology modifier polymers such as, for example, Acrylates Steareth-20 Methacrylate Copolymer, Acrylates Beheneth-25 Methacrylate Copolymer, Acrylates Steareth-20 Methacrylate Crosspolymer, Acrylates Copolymer, Acrylates/Vinylneodecanoate Crosspolymer, and mixtures thereof.

In one embodiment, the personal care composition, is heat stable up to at least 60° C.

In one embodiment, the personal care composition is a hair care composition. In such embodiments, the methylcellulose ether is present in a range from 0.1 wt % to 10 wt % by weight of the personal care composition, preferably in a range from 0.5 wt % to 5.0 wt %.

In one embodiment, the hair care composition is a hair fixative. In one embodiment, the hair fixative polymer is at least one of PVP/VA copolymer, ethyl ester of PVM/MA copolymer, butyl ester of PVM/MA copolymer, vinyl acetate/crotonic acid copolymer, vinyl acetate/crotonic acid/vinyl neodecanoate, VA/butyl maleate/isobornyl acrylate copolymer, acrylates copolymer, diglycol/CHDM/isophthalates/SIP copolymer, acrylates/hydroxyester acrylates copolymer, methacrylates/acrylates copolymer/amine salt, AMP-acrylates/diacetone-acrylamide copolymer, AMPD-acrylates/diacetone-acrylamide copolymer, acrylates/methacrylate polymers, acrylates/acrylamide copolymer, PVP/vinyl caprolactam/DMAPA acrylates copolymer, polyvinylcaprolactam, isobutylene/ethylmaleimide/hydroxyethylmaleimide copolymer, acrylates/succinates/hydroxyacrylates copolymer, polyurethane-1, Octylacrylamide/Acrylates/Butylaminoethyl Methacrylate Copolymer, Vinyl caprolactam/VP/Dimethylaminoethyl Methacrylate Copolymer, Acrylates/t-Butylacrylamide Copolymer, Acrylates/C1-2 Succinates/Hydroxyacrylates Copolymer, Acrylamide/Sodium Acryloyldimethyltaurate/Acrylic Acid Polymer and mixtures thereof.

In one embodiment, the personal care composition is a skin care composition. In such embodiments, the methylcellulose ether is present in a range from 0.1 wt % to 70 wt % by weight of the personal care composition, preferably in a range from 5 wt % to 40 wt %. In one embodiment, the personal care composition contains an antioxidant, anti-aging active, or sun care active.

In one embodiment, the personal care composition is formulated as an emulsion, gel or other aqueous based formulation, to enable a stable, airy, whipped texture at room temperature, preferentially displaying structural stability over a broad range of high temperature exposure. Personal care products of the present invention exhibit a texture that can be described as airy, whipped, fluffy, and/or light. As can be appreciated, the texture of personal care formulations can have a major influence on consumer perception of product performance. For example, products with an airy, light texture will be perceived by consumers to improve hair volume or to result in faster absorbance into skin. Moreover, personal care products of the present invention exhibit marked ability to maintain their airy, light emulsion structure during product transportation and shelf-life storage.

In one embodiment, the present invention is a facial mask help to cleanse, condition and soothe the skin. Skin treatments are designed to place an active ingredient at a specific location for a long enough period of time to be effective. These masks and treatments, however, can be difficult to apply because they are either very viscous, or alternatively, they may he too thin and not hold their shape very well. Previously, facial masks used high levels of filler compounds such as Kaolin clay to help hold the mask in place, which made it more difficult for beneficial agents to penetrate to the skin surface, as well as difficult to apply and spread, or otherwise imparted poor overall aesthetics. Likewise, consumers often had to lie still throughout the application process and over the length of time required the mask or treatments to work. In contrast, low gelation temperature methylcellulose allows for formulation of a low viscosity mask or treatment that is easy to apply and will gel on skin, giving significantly better hold on skin than when conventional methylcellulose.

In some embodiments, the personal care composition includes an emollient. The emollient may be at least one of moisturizer, conditioner, oil, or other fatty substance. For example, when the composition is in an emulsion form, it comprises at least one oily phase that contains at least one oil, especially a cosmetically acceptable oil. The term “oil” means a fatty substance that is liquid at room temperature.

Examples of oils include hydrocarbon-based oils of animal origin, such as squalene, hydrocarbon-based oils of plant origin, such as liquid triglycerides of fatty acids comprising from 4 to 10 carbon atoms, for instance heptanoic or octanoic acid triglycerides, or alternatively, oils of plant origin, for example sunflower oil, corn oil, soybean oil, marrow oil, grapeseed oil, sesame seed oil, hazelnut oil, apricot oil, macadamia oil, arara oil, coriander oil, castor oil, avocado oil, jojoba oil, shea butter oil, or caprylic/capric acid triglycerides, MIGLYOL 810, 812 and 818 (from Dynamit Nobel), synthetic esters and ethers, especially of fatty acids, for instance the oils of formulae R¹COOR² and R¹OR² in which le represents a fatty acid residue comprising from 8 to 29 carbon atoms and R² represents a branched or unbranched hydrocarbon-based chain comprising from 3 to 30 carbon atoms, for instance purcellin oil, isononyl isononanoate, isopropyl myristate, 2-ethylhexyl palmitate, 2-octyldodecyl stearate, 2-octyldodecyl erucate or isostearyl isostearate, hydroxylated esters, for instance isostearyl lactate, octyl hydroxystearate, octyldodecyl hydroxystearate, diisostearyl malate, triisocetyl citrate and fatty alcohol heptanoates, octanoates and decanoates, polyol esters, for instance propylene glycol dioctanoate, neopentyl glycol diheptanoate and diethylene glycol diisononanoate, pentaerythritol esters, for instance pentaerythrityl tetraisostearate, lipophilic derivatives of amino acids, such as isopropyl lauroyl sarcosinate, such as is sold under the name ELDEW SL 205 (from Ajinomoto), linear or branched hydrocarbons of mineral or synthetic origin, such as mineral oils (mixtures of petroleum-derived hydrocarbon-based oils), volatile or non-volatile liquid paraffins, and derivatives thereof, petroleum jelly, polydecenes, isohexadecane, isododecane, hydrogenated isoparaffin (or polyisobutene), silicone oils, for instance volatile or non-volatile polymethylsiloxanes (PDMS) comprising a linear or cyclic silicone chain, which are liquid or pasty at room temperature, especially cyclopolydimethylsiloxanes (cyclomethicones) such as cyclopentasiloxane and cyclohexadimethylsiloxane, polydimethylsiloxanes comprising alkyl, alkoxy or phenyl groups, which are pendent or at the end of a silicone chain, these groups comprising from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes 2-phenylethyltrimethyl siloxysilicates and polymethylphenylsiloxanes, fluoro oils such as partially hydrocarbon-based and/or partially silicone-based fluoro oils, ethers such as dicaprylyl ether (CTFA name: dicaprylyl ether), and C₁₂-C₁₅ fatty alcohol benzoates (FINSOLV TN from Finetex), mixtures thereof.

Oils include mineral oil, lanolin oil, coconut oil and derivatives thereof, cocoa butter, olive oil, almond oil, macadamia nut oil, aloe extracts such as aloe vera lipoquinone, jojoba oils, safflower oil, corn oil, liquid lanolin, cottonseed oil, peanut oil, hydrogenated vegetable oil, squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, and the oil of cereal germs.

Other suitable emollients include dicaprylyl ether, C₁₂₋₁₅ alkyl benzoate, DC 200 FLUID 350 silicone fluid (from Dow Corning Corp.), isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, phenyltrimethicone, and aloe vera extract. Solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate.

In one embodiment, the emollient is present in an amount from 0.05% to 40% by weight of the composition. Preferably, the emollient is present in an amount from 0.1% to 10% by weight of the composition.

In some embodiments, the personal care composition includes an emulsifier or a surfactant. Suitable emulsifiers are selected from amphoteric, anionic, cationic and nonionic emulsifiers, used alone or as a mixture. Anionic surfactants include soaps or salts of fatty acids, alkyl sulfates, alkyl ether sulfates, alpha-olefin sulfonates, alkyl aryl sulfonates, sarcosinates, alkyl glucose esters or their alkoxylates, and in particular sodium lauryl sulfate, ammonium lauryl sulfate, triethanolamine lauryl sulfate, sodium laureth sulfate, isethionates, and triethanolamine stearate. Nonionic surfactants include methyl glucose stearates or their ethoxylates, alkyl polyglucosides, and glycerol monostearate, fatty acid alkanol amides, alkyl aryl polyglycol ether, polyglycol ethers and in particular cocoyl diethanolamide, nonoxynol-7 and octoxynol-9; cationics including alkyl trimethyl ammonium salts, quaternized amides of ethylene diamine, alkyl pyridinium salts and in particular cetrimonium chloride, stearalkonium chloride and cetyl pyridinium chloride; and amphoterics including alkyl .beta.-aminopropionates, betaines, alkyl imidazolines and in particular cocamidopropyl betaine and caproam phocarboxy propionate. Polymeric cationic emulsifiers that include hydrophobic moieties are preferred, examples of which include polyquaternium-24 and polyquaternium 67 (SOFTCAT™), available from The Dow Chemical Company.

Emulsions free of emulsifying surfactants or comprising less than 0.5% of emulsifying surfactants relative to the total weight of the composition may also be prepared, by using suitable compounds, for example polymers having emulsifying properties, such as CARBOPOL 1342 polymer (Noveon), PEMULEN polymer (Noveon), SEPIGEL 305 polyacrylamide/C13-C14 isoparaffin/laureth-7 (Seppic), particles of ionic or nonionic polymers, particles of anionic polymer such as, isophthalic acid, sulfoisophthalic acid polymers, and phthalate/sulfoisophthalate/glycol copolymers (for example diethylene glycol/phthalate/isophthalate/1,4-cyclohexanedimethanol sold under the names Eastman AQ diglycol/CHDM/isophthalates/SIP copolymer (AQ35S, AQ38S, AQ55S and/or AQ48 Ultra, from Eastman Chemical). Emulsifier-free emulsions stabilized with silicone particles or metal oxide particles such as TiO₂ or the like may also be prepared.

The emulsifier or surfactant may be present in an amount from 0.01% to 15% by weight of the composition. In one embodiment, the surfactant is present in an amount from 0.1% to 5% by weight of the composition.

In some embodiments, the personal care composition includes a thickener. Examples of thickeners include polymers, for example, modified or unmodified carboxyvinyl polymers, such as the products sold under the names CARBOPOL and PEMULEN (INCI name: Acrylates/C 10-30 alkyl acrylate crosspolymer; available from Noveon), polyacrylates and polymethacrylates, such as the products sold under the names LUBRAJEL and NORGEL (from Guardian) or HISPAGEL (from Hispano Chimica), polyacrylamides, 2-acrylamido-2-methylpropanesulfonic acid polymers and copolymers, which are optionally crosslinked and/or neutralized, for instance the poly(2-acrylamido-2-methylpropane-sulfonic acid) sold by Clariant (INCI name: ammonium polyacryldimethyltauramide), emulsified crosslinked anionic copolymers of acrylamide and AMPS, such as those sold under the name SEPIGEL 305 (INCI name: Polyacrylamide/C13-14 Isoparaffin/Laureth-7; from Seppic) and under the name SIMULGEL 600 (INCI name: Acrylamide/Sodium acryloyldimethyltaurate copolymer/Isohexadecane/Polysorbate 80; from Seppic), polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose, associative polymers, for instance associative polyurethanes, copolymers comprising at least two hydrocarbon-based lipophilic chains comprising from 6 to 30 carbon atoms, separated with a hydrophilic sequence, such as the polyurethanes sold under the names SERAD FX1010, SERAD FX1100 and SERAD FX1035 (from Hüls America), RHEOLATE 255, RHEOLATE 278 and RHEOLATE 244 (INCI name: Polyether-urea-polyurethane; from Rheox), DW 1206F, DW 1206J, DW 1206B, DW 1206G, and ACRYSOL RM 2020 (from Röhm & Haas), Aculyn 44, Aculyn 46 (from The Dow Chemical Company). sodium magnesium silicate, for instance Laponite XLG. One preferred thickener is METHOCEL hydroxypropyl methylcellulose, available from The Dow Chemical Company.

In one embodiment, the thickener is present in an amount from 0.01% to 10% by weight of the composition. In one embodiment, the thickener is present in an amount from 0.1% to 5% by weight of the composition.

The personal care composition also comprises a suitable carrier, or mixtures of carriers. The type of carrier depends on the particular end use of the composition. Illustrative carriers include, for example, water, such as deionized or distilled water, emulsions, such as oil-in-water or water-in-oil emulsions, alcohols, such as ethanol, isopropanol or the like, glycols, such as propylene glycol, glycerine or the like, or combinations thereof. A preferred carrier is deionized water.

In one embodiment, the personal care compositions of the present invention further comprise an active ingredient selected from skin care actives, nail care actives, or hair care actives. Actives include sunscreens, skin colorants, drug substances (such as anti-inflammatory agents, antibiotics, topical anesthetics, antimycotics, keratolytics, and the like), skin protectants, conditioners, humectants, and ultraviolet radiation absorbers.

Examples of sunscreens include para aminobenzoic acid, avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, trolamine salicylate, titanium dioxide, zinc oxide, benzophenones, benzylidenes, salicylates, or other known UV filters, including diethanolamine methoxycinnamate, digalloy trioleate, ethyl dihydroxypropyl PABA, glyceryl aminobenzoate, and lawsone with dihydroxy acetone and red petrolatum.

In one embodiment, the personal care compositions of the present invention further comprise at least one additional ingredient. Optional ingredients include any suitable substance for personal care compositions, for example, colorants, preservatives, pH adjustors, reducing agents, fragrances, foaming agents, tanning agents, depilatory agents, astringents, antiseptics, deodorants, antiperspirants, insect repellants, and biocides.

Colorants include pigments, which are used especially in make-up, including metal oxide pigments, titanium dioxide, optionally surface-treated, zirconium oxide or cerium oxide, zinc oxide, iron oxide (black, yellow or red), chromium oxide, manganese violet, ultramarine blue, chromium hydrate and ferric blue, carbon black, pigments of barium, strontium, calcium or aluminum (for example D&C or FD&C), cochineal carmine, mica coated with titanium or with bismuth oxychloride, titanium mica with iron oxides, titanium mica with, especially, ferric blue or chromium oxide, titanium mica with an organic pigment, nacreous pigments based on bismuth oxychloride, goniochromatic pigments, for example pigments with a multilayer interference structure, reflective pigments, for example particles with a silver-coated glass substrate, glass substrate coated with nickel/chromium/molybdenum alloy, glass substrate coated with brown iron oxide, particles comprising a stack of at least two polymer layers, for instance MIRROR GLITTER (from 3M).

Dyes include water-soluble dyes such as copper sulfate, iron sulfate, water-soluble sulfopolyesters, rhodamines, natural dyes, for instance carotene and beetroot juice, methylene blue, caramel, the disodium salt of tartrazine and the disodium salt of fuschin, and mixtures thereof. Liposoluble dyes may also optionally be used.

Preservatives include alcohols, aldehydes, methylchloroisothiazolinone and methylisothiazolinone, p-hydroxybenzoates, and in particular methylparaben, propylparaben, glutaraldehyde and ethyl alcohol.

The pH adjustors, include inorganic and organic acids and bases and in particular aqueous ammonia, citric acid, phosphoric acid, acetic acid, sodium hydroxide, potassium hydroxide, and triethanolamine. In a preferred embodiment, the pH adjustor is aminomethyl propanol, L-arginine, tromethamine, PEG-15 cocamine, diisopropanolamine, triisopropanolamine, or tetrahydroxypropyl ethylenediamine. In a particularly preferred embodiment, the pH adjustor is amino methyl propanol, Aminomethyl propanol is available under the tradename AMP-ULTRA from Angus Chemical Company. In one embodiment, the pH adjustor is present in an amount from 0.01% to 1% by weight of the composition. In one embodiment, the pH adjustor is present in an amount from 0.1% to 0.5% by weight of the composition.

Reducing agents include ammonium thioglycolate, hydroquinone and sodium thioglycolate.

Fragrances include any component which provides a pleasant scent. Fragrances are generally aldehydes or ketones, and often oils obtained by extraction of natural substances or synthetically produced. Often, fragrances are accompanied by auxiliary materials, such as fixatives, extenders, stabilizers and solvents.

Biocides include antimicrobials, bactericides, fungicides, algaecides, mildicides, disinfectants, antiseptics, and insecticides.

The amount of optional ingredients effective for achieving the desired property provided by such ingredients can be readily determined by one skilled in the art.

In one embodiment, the personal care composition may be formulated in the form of a leave-on hair composition, containing a moisturizer, conditioner, and/or styling active.

In one embodiment, the personal care composition may be formulated in the form of a skin care composition, such as a lotion or cream, containing a moisturizer, anti-aging, and/or suncare active. In one embodiment, the personal care composition may be formulated in the form of a skin care composition, such as a mask.

### EXAMPLES

The following examples are for illustrative purposes only and are not intended to limit the scope of the present invention. All percentages are by weight unless otherwise specified.

### Example 1

An example of a personal care composition of the present invention is listed in TABLE 1, in wt %:

TABLE 1

| | | Batch A |
|---|---|---|
| A | DI water | 69.3 |
| | SGA7C Methylcellulose (The Dow Chemical | 0.5 |
| | Company); gelation temperature ~45° C. | |
| | Acrylates/Hydroxyester acrylates copolymer | 4.0 |
| | (40% solids) | |
| B | DI water | 20.0 |
| | Acrylate/Steareth-20 Methacrylate Crosspolymer | 2.0 |
| | (29% solids) | |
| C | Stepan Stearyl Alcohol | 1.0 |
| | Stepan Cetyl Alcohol | 2.0 |
| | PEG-40 hydrogenated castor oil | 0.1 |
| D | Aminomethyl proponal | 0.4 |
| E | Propylene Glycol | 0.2 |
| | Vitamin E acetate USP | 0.01 |
| | Methylisothiazolinone, Phenoxyethanol | 0.45 |

To prepare the formulation, the DI water from Part A is heated to 80-90° C., and SGA7C methylcellulose powder sprinkled in and stirred. Once a consistent dispersion is formed, reduce the temperature to 70-75° C., add remaining of Part A components. The Part B components are then added to Part A and mixed, while maintaining the temperature at 70-75° C.

The Part C components are combined in a separate vessel, and heated slowly to liquefy at 70-75° C. The Part C phase is then slowing added to the Part A&B mixture, and mixed at high speed for 5 minutes while maintaining the temperature. The heat is then removed, and Part D added before the formulation cools.

The resulting formulation is allowed to air cool to 40° C., and Part E is added, and the formulation mixed for 15 minutes.

### Example 2

### Comparative

An example of a comparative personal care composition is listed in TABLE 2, in wt %:

TABLE 2

| | | Comparative |
|---|---|---|
| | | Batch 1 |
| A | DI water | 69.3 |
| | A4M Methylcellulose (The Dow Chemical | 0.5 |
| | Company); gelation temperature ~65° C. | |
| | Acrylates/Hydroxyester acrylates copolymer | 4.0 |
| | (40% solids) | |
| B | DI water | 20.0 |
| | Acrylate/Steareth-20 Methacrylate Crosspolymer | 2.0 |
| | (29% solids) | |
| C | Stepan Stearyl Alcohol | 1.0 |
| | Stepan Cetyl Alcohol | 2.0 |
| | PEG-40 hydrogenated castor oil | 0.1 |
| D | Aminomethyl proponal | 0.4 |
| E | Propylene Glycol | 0.2 |
| | Vitamin E acetate USP | 0.01 |
| | Methylisothiazolinone, Phenoxyethanol | 0.45 |

The formulation is prepared substantially similar to Example 1.

### Example 3

Examples of personal care compositions of the present invention are listed in TABLE 3, in wt %:

TABLE 3

| | | Batch B |
|---|---|---|
| A | DI water | 61.7 |
| | SGA7C Methylcellulose (The Dow Chemical | 0.5 |
| | Company); gelation temperature ~45° C. | |
| | Acrylates/Hydroxyester acrylates copolymer | 4.0 |
| | (45% solids) | |
| B | DI water | 20.0 |
| | Sodium Magnesium Silicate | 0.1 |
| | Acrylate/Steareth-20 Methacrylate Crosspolymer | 2.0 |
| | (29% solids) | |
| C | Cocoamido betaine | 0.5 |
| | Cetearyl Isononanoate, Ceteareth-20, Cetearyl | 5.0 |
| | Alcohol, Glyceryl Stearate, Glycerin, Ceteareth-12, | |
| | Cetyl Palmitate | |
| | Pentaerythrityl Tetracaprylate/Tetracaprate | 5.0 |
| | PEG-40 hydrogenated castor oil | 0.1 |
| D | AMP-95 Aminomethyl proponal | 0.4 |
| E | Propylene Glycol | 0.2 |
| | Methylisothiazolinone, Phenoxyethanol | 0.45 |

The formulations are prepared substantially similar to Example 1, except a 1% stock solution of SGA7C was first prepared by dispersing in hot water 80-90° C. After a consistent dispersion is formed, cool the solution to 8-10° C. 50g of the 1% SGA7C stock solution is added into phase A.

### Example 4

To test aesthetic desirability, Batches A & B, and Comparative Batch 1 from the previous examples were made. Upon evaluation by a trained panelist, Batch A was characterized as a whipped yogurt-like texture, Batch B was characterized as an “airy lotion,” whereas Comparative Batch 1 was characterized as a “waxy emulsion,” which is aesthetically unacceptable. METHOCEL A4M Methyl Cellulose is a known thickener in many application areas to modify the flow and rheology properties; accordingly, the exemplary texture of the inventive samples cannot be attributed to mere thickening ability.

Batch B was tested conventionally and showed good heat aging stability with no phase separation, maintaining a fluffy, airy lotion texture after a 10 day, 60° C. heat aging stability test. The formulation was stored in a clear glass jar, sealed with plastic screw cap. The glass container was then stored in 60° C. oven for 10 days. At the end of the testing cycle, the sample was equilibrated at room temperature. Visual observation on formulation appearance and phase separation was made to determine formulation heat-aging stability.

### Example 5

An example of a personal care composition of the present invention is listed in TABLE 4, in wt %:

TABLE 4

| | | Comparative |
|---|---|---|
| | Batch C | Batch 2 |
| DI water | 49.5 | 49.5 |
| SG A7C Methylcellulose (The Dow Chemical | 50 | — |
| Company); gelation temperature ~45° C. | | |
| (2% soln) | | |
| A4M Methylcellulose (The Dow Chemical | — | 50 |
| Company); gelation temperature ~65° C. | | |
| (2% soln) | | |
| Polyquaternium-24 (and) Hyaluronic acid | 0.5 | 0.5 |
| (BIOCARE ™ HA-24) | | |

Components are combined to form a face mask. Many skin treatments include the application of a composition to the face, followed by a leave on period of several minutes to an hour. In order to spread the product onto the face, the viscosity must be low enough for ease of use. This, however, can cause the product to slide off the face during the leave on period.

Batch C and Comparative Batch 2 were prepared and placed in Petri dishes. At room temperature, both compositions ran when the dish was tilted. However, when the dishes were heated to 38° C., Batch C ran significantly slower than Comparative Batch 2, which showed no change from room temperature viscosity.

Batch C and Comparative Batch 2 were prepared and placed on separate pieces of pre-wetted Vitro-Skin placed on a panelist's forearm. After waiting for 1-2 min, the panelist elevated the forearm. Comparative Batch 2 ran as soon as the panelist started bringing up the forearm. In contrast, Batch C did not run down the forearm, even at higher angles of elevation.

### Example 6

An example of a personal care composition of the present invention is listed in TABLE 5, in wt %:

TABLE 5

| | | Batch D |
|---|---|---|
| A | DI water | 67.2 |
| | SG A7C Methylcellulose (The Dow Chemical | 25 |
| | Company); gelation temperature ~45° C. (1% soln) | |
| | AMP-95 Aminomethyl proponal | 0.4 |
| | ACUDYNE 1000 Acrylates/Hydroxyester | 4.0 |
| | acrylates copolymer (45% solids) | |
| | ACULYN 88 Acrylate/Steareth-20 Methacrylate | 2.0 |
| | Crosspolymer (29% solids) | |
| B | Propylene Glycol | 0.2 |
| | PEG-40 hydrogenated castor oil | 0.1 |
| | Laureth-4 | 0.1 |
| | Cocoamidopropyl betaine | 0.5 |
| | NEOLONE PE Methylisothiazolinone, | 0.45 |
| | Phenoxyethanol | |

To prepare the formulation, a pre-solution of methylcellulose is created. The phase A ingredients are dispersed together in water. The phase B ingredients are mixed with the dispersed phase A solution, and mixed until uniformed solution is formed. The solution is loaded in an aerosol can (96% of the liquid formulation charged under pressure with 6% hydrocarbon propellants), which dispenses the formulation as a mousse.

### Example 7

### Comparative

An example of a personal care composition of the present invention is listed in TABLE 6, in wt %:

TABLE 6

| | | Comparative |
|---|---|---|
| | | Batch 3 |
| A | DI water | 79.7 |
| | A4M Methylcellulose (The Dow Chemical | 12.5 |
| | Company); gelation temperature ~65° C. (2% soln) | |
| | AMP-95 Aminomethyl proponal | 0.4 |
| | ACUDYNE 1000 Acrylates/Hydroxyester | 4.0 |
| | acrylates copolymer (45% solids) | |
| | ACULYN 88 Acrylate/Steareth-20 | 2.0 |
| | Methacrylate Crosspolymer (29% solids) | |
| B | Propylene Glycol | 0.2 |
| | PEG-40 hydrogenated castor oil | 0.1 |
| | Laureth-4 | 0.1 |
| | Cocoamidopropyl betaine | 0.5 |
| | NEOLONE PE Methylisothiazolinone, | 0.45 |
| | Phenoxyethanol | |

The formulation is prepared substantially similar to Example 6.

### Example 8

To test aesthetic desirability, Batch D and Comparative Batch 3 from the previous examples were made. Upon evaluation by a trained panelist, Batch D showed good initial foam density and stability, whereas Comparative Batch 3 showed poor initial foam density and stability, which is aesthetically unacceptable. METHOCEL A4M Methyl Cellulose is a known thickener; accordingly, the exemplary texture of the inventive samples cannot be attributed to mere thickening ability.

Upon application to commercially available hair swatches, followed blow-drying, Batch D showed that hair volume increased significantly (Dia-Stron Miniature Tensile Tester 175; higher % change in combing force).

To study the foam morphology, a thin layer of foam from Batch D was cast on a black tile substrate. Batch D generated a much denser and creamier foam upon actuation out of the aerosol can than a comparative commercial mousse. Upon blow-drying for 1 minute, the comparative commercial mousse collapsed instantly, and formed large areas with an absence of film. In contrast, Batch D maintained a dense foam cell structure. Without being bound by theory, this performance should boost hair volume by maintaining a stable, uniform, supportive structure on hair.

To better characterize the mechanism of mousse foam stability, the extensional viscosity of fully formulated products designed for a mousse application was measured at room temperature and 50° C., to simulate application and heat activation via blow-drying. The solutions (each at a concentration of 0.25% methylcellulose per 100 g solids) were studied in a Haake Capillary Breakup Extensional Rheometer™ (CABER1). The plate diameter was 6.0 mm and the gap was 3.0 mm The cushioned profile was used at a strike time of 50 ms and a final height of 18.75 mm. A nominal surface tension of 30 mN/m and a density of 1000 kg/m3 were input to the program, as the exact values are not known for all the solutions and relative comparisons are sought. For the 50° C. temperature, after the sample was loaded it was allowed to equilibrate for 45 sec before starting the test.

Overall, the filament break-up times seemed to correlate well with observed film formation properties of the mousse before and after heat treatment. Batch D and Comparative Batch 3 showed similar behavior at room temperature (filament breakup times at 0.03 and 0.04 seconds, respectively). However, at 50° C., Batch D gelled, which shows an increase in strength/breakup time (greater than 1.0 second), versus Comparative Batch 3 showed a decrease in breakup time at elevated temperature (filament breakup times at 0.02 seconds).

1. A personal care composition, comprising:
water;
a methylcellulose that gels at 45° C. or less; and
at least one one hair fixative polymer, moisturizer, conditioner, humectant, cationic conditioning polymer, anti-aging active, or sun care active.

2. The personal care composition of claim 1, further comprising one or more rheology modifier polymers such as, for example, Acrylates Steareth-20 Methacrylate Copolymer, Acrylates Beheneth-25 Methacrylate Copolymer, Acrylates Steareth-20 Methacrylate Crosspolymer, Acrylates Copolymer, Acrylates/Vinylneodecanoate Crosspolymer, and mixtures thereof.

3. The personal care composition of claim 1, wherein the personal care composition is a hair care composition.

4. The personal care composition of claim 3, wherein the methylcellulose ether is present in a range from 0.1 wt % to 10 wt % by weight of the personal care composition, preferably in a range from 0.5 wt % to 5.0 wt %.

5. The personal care composition of claim 3, wherein the hair fixative polymer is at least one of PVP/VA copolymer, ethyl ester of PVM/MA copolymer, butyl ester of PVM/MA copolymer, vinyl acetate/crotonic acid copolymer, vinyl acetate/crotonic acid/vinyl neodecanoate, VA/butyl maleate/isobornyl acrylate copolymer, acrylates copolymer, diglycol/CHDM/isophthalates/SIP copolymer, acrylates/hydroxyester acrylates copolymer, methacrylates/acrylates copolymer/amine salt, AMP-acrylates/diacetone-acrylamide copolymer, AMPD-acrylates/diacetone-acrylamide copolymer, acrylates/methacrylate polymers, acrylates/acrylamide copolymer, PVP/vinyl caprolactam/DMAPA acrylates copolymer, polyvinylcaprolactam, isobutylene/ethylmaleimide/hydroxyethylmaleimide copolymer, acrylates/succinates/hydroxyacrylates copolymer, polyurethane-1, Octylacrylamide/Acrylates/Butylaminoethyl Methacrylate Copolymer, Vinyl caprolactam/VP/Dimethylaminoethyl Methacrylate Copolymer, Acrylates/t-Butylacrylamide Copolymer, Acrylates/C1-2 Succinates/Hydroxyacrylates Copolymer, Acrylamide/Sodium Acryloyldimethyltaurate/Acrylic Acid Polymer and mixtures thereof.

6. The personal care composition of claim 3, wherein the personal care composition further comprises propellants.

7. The personal care composition of claim 1, wherein the personal care composition is a skin care composition.

8. The personal care composition of claim 7, wherein the methylcellulose ether is present in a range from 0.1 wt % to 70 wt % by weight of the personal care composition, preferably in a range from 5 wt % to 40 wt %.

9. The personal care composition of claim 7, wherein the personal care composition contains an antioxidant, anti-aging active, or sun care active.

10. A method for inducing volume in hair, comprising:
applying to the hair the personal care composition of claim 3; and
heat drying the hair.
